(19) United States
(12) Patent Application Publication
(10) Pub. No.: US 2004/0235152 A1
(43) Pub. Date: Nov. 25, 2004

(54) Apparatus for and methods of in vitro fertilization

(76) Inventor: Farhang Abed, Pasdaran (IR)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/481,544

(22) PCT Filed: Jun. 18, 2002
(86) PCT No.: PCT/GB02/02789

(30) Foreign Application Priority Data
Jun. 20, 2001 (GB) 0115104.2

(51) Int. Cl. C12M 1/00

(52) U.S. Cl. 435/289.1

(57) ABSTRACT

A multi-chamber module for use in the insemination of oocytes comprises a first chamber (36) for holding a fluid medium, a second chamber (54) for receiving semen, and a third chamber (52) into which the fluid medium is arranged to flow from the first chamber (36) and from which that fluid medium passes into the second chamber (54), thereby to permit sperm to move against the fluid flow and pass by capillary flow from the second chamber (54) into the third chamber (52) where the insemination takes place. The capillary flow is established in radial passages (51) between the confronting surfaces of cup-shaped containers (24) and 40). A waste chamber (38) surrounds the second chamber (54).

The present invention relates generally to infertility, and particularly to apparatus for and methods of in vitro fertilization (IVF). More particularly, the invention is concerned with effecting sperm isolation, insemination and culture in a single device.

### BACKGROUND OF THE INVENTION

In standard human IVF procedures, a 16-hour incubation of oocytes with prepared spermatozoa in a drop of medium and under mineral oil was originally established for practical reasons. This generally corresponds to the time for observations for pronuclei. However, the overnight coincubation of oocytes with sperm is not physiological and sperm metabolic waste products may have detrimental effects on the zygote, including zona hardening, thus impairing implantation and pregnancy rates.

The most commonly used sperm preparation techniques for IVF have included “Swim up” and “Percol”, that involve chemicals, washing and centrifugation. These techniques may result in some damage to the sperm. Aitken and Clarkson have shown that centrifugal force generates the production of reactive oxygen species that may damage sperm and impair their fertilization potential. (R. J. Aitken and J. S. Clarkson “J. Reprod. Fertil.” 1991;6: pages 173-176). “Percol” has been used largely in the setting of laboratory research and its clinical use is associated with certain disadvantages. Some batches have been found to contain high levels of endotoxin, making them unsuitable for clinical use (C. Y. Andersen and J. Grinsted: “J. Assisted Reprod. Gent.” 1997;14: pages 624-628). In late 1996, “Percol” was withdrawn from clinical use as a sperm separation medium (Guneet Makkar et al. “Fertil. Steril.” 1999;72: pages 796-802).

It has been reported that when sperm are put into a fluid flow, the motile sperm rapidly align themselves and swim against the flow (F. Abed. “The new finding of a phenomenon in sperm motility: the spermatozoa swims against flow”—from “In vitro fertilization and assisted reproduction”, edited by V. Gomel and P. C. K. Leung, Monduzzi Editore, 1997: pages 13-15). Non-motile and sluggish sperm, along with other cellular components, are washed downstream away from the motile sperm. Cilia have been shown to be present in endometrial cells of many mammals. Ciliary currents in both the fallopian tubes and the uterus move in the same direction and extend towards the external os. One may expect that this flow performs two functions. Firstly, this flow acts as a guide for sperm, leading sperm with the correct motility parameters towards the site of fertilization at the ampoule of the fallopian tubes. Secondly, this flow acts as a natural selection mechanism to optimize the quality of sperm able to reach the fertilization site.

### SUMMARY OF THE INVENTION

It is an object of the present invention to utilise the known phenomenon of sperm alignment against flow in order to be able both to prepare sperm for assisted reproductive techniques and procedures, in particular IVF procedures, and also to achieve insemination, in the one vessel.

In accordance with the present invention there is provided an apparatus for the insemination of oocytes comprising a multi-chamber module having a first chamber for holding a fluid medium, a second chamber for receiving semen, and a third chamber into which the fluid medium is arranged to flow from the first chamber and from which that fluid medium is arranged to pass into the second chamber, thereby to permit sperm to move against the fluid flow and pass from the second chamber into the third chamber where insemination of oocytes is arranged to take place.

In a preferred embodiment, the second and third chambers are separated by barrier means through which fluid is arranged to flow from the third chamber to the second chamber and through which sperm are arranged to pass from the second chamber to the third chamber.

This barrier can comprise a passageway or passageways between the chambers.

The module also preferably includes control means for controlling the velocity of flow of the fluid from the first chamber to the third chamber.

In a preferred embodiment, the second and third chambers are defined by cup-shaped containers, one within the other and with the chambers separated by an annular flow-restricting barrier through which the fluid is arranged to flow.

Preferably, the module includes a waste chamber into which waste material can pass from the second chamber, for example via filter means.

Also in accordance with the present invention there is provided a method of preparing sperm for insemination, comprising establishing within a multi-chamber module a flow of fluid medium from a first chamber to a second chamber via a third chamber by filling the first chamber with the fluid medium, adding oocytes to the third chamber, and adding semen to the second chamber, whereby motile sperm can pass, against the fluid flow, from the second chamber to the third chamber to inseminate the oocytes therein.

Preferably, the flow of fluid medium from the first chamber to the second chamber is controlled so that a steady-state flow of fluid medium is arranged to pass from the third chamber to the second chamber.

Continued culture of the inseminated oocytes can take place within the third chamber.

Also in accordance with the present invention there is provided an IVF method which comprises effecting within a single multi-chamber module separation of motile sperm and the insemination of oocytes by the separated motile sperm.

Preferably, the method includes subsequent culture within the same module.

The apparatus and methods of the present invention have a number of advantages over conventional methods. The present invention does not induce any damage to the sperm, because the procedure does not require any use of chemicals or centrifuges. The preparation process is rapid and simple. The process of sperm separation is under direct observation and can easily be controlled. The module can be used by physicians without the need for laboratory equipment. Also, the use of the module in accordance with the invention not only serves to separate sperm, but also washes the sperm, thus eliminating the need for any centrifuge process.

### BRIEF DESCRIPTION OF THE DRAWING

A more detailed description of the present invention will now be given, with reference to the accompanying drawing, wherein like reference characters refer to like parts throughout the several views, and in which:

FIG. 1 is a partially cut-away sectional view of a preferred embodiment of insemination module in accordance with the invention;

FIG. 2 is a view, on an enlarged scale, of the fluid flow control means of the module of FIG. 1; and

FIG. 3 is a view, again on an enlarged scale, of the passage between the seminal chamber and the insemination chamber of the module shown in FIG. 1.

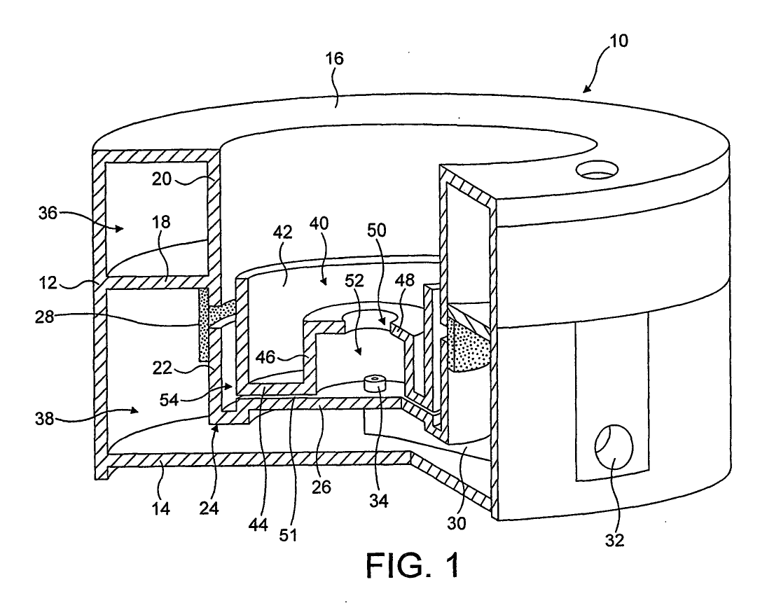

### DESCRIPTION OF THE PREFERRED EMBODIMENT

Referring to the drawing, the multi-chamber module of the present invention is indicated generally at 10. The module is substantially cylindrical in shape, with an outer circumferential wall 12, a base 14 and a top wall 16. It is preferably made of plastics material. Extending radially inwardly from the outer circumferential wall 12 is a horizontal dividing wall 18 which continues at its radially inner face as an upwardly extending vertical wall 20 which joins the top wall 16. Vertically below the wall 20 is an outer circumferential wall 22 of a cup-shaped container indicated generally at 24. This container 24 has a base 26 which is stepped around the periphery. The upper edge of the outer wall 22 of the container 24 is spaced from the bottom of the vertical wall 20 to define a circumferential slot therebetween. This slot is plugged by a membrane filter 28. The filter is preferably such as to permit the passage only of material below a figure within the range of 1.3 to 3.5 microns, preferably below 3 microns. The cup-shaped container 24 is supported within the module, for example on a support member 30 which is set on the base 14 of the module. The interior of the support member 30 connects with a port 32 in the exterior wall 12 of the module.

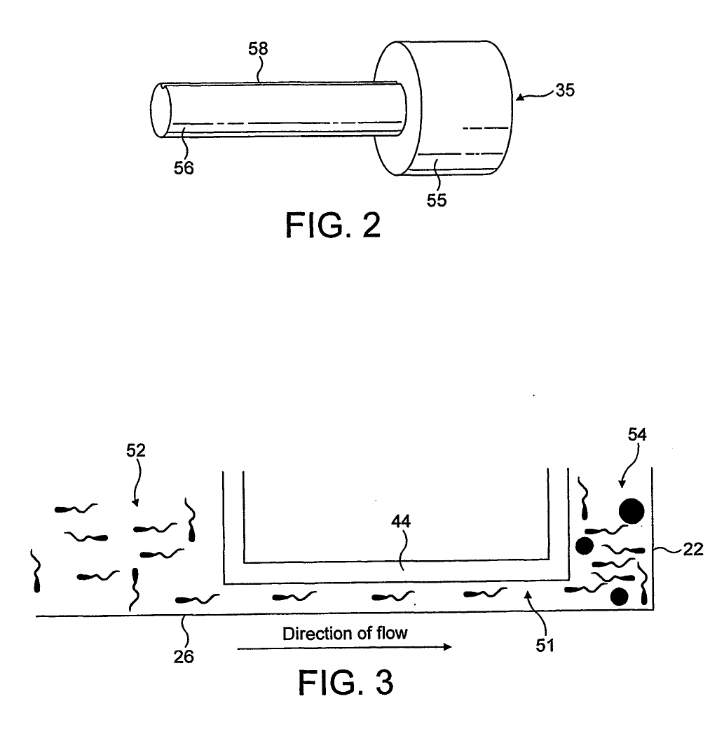

A hole 34 is formed through the base 26 of the container 24, adjacent to its centre. Hole 34 communicates with outlet port 32 via a valve 35 (FIG. 2). The valve associated with outlet port 32 is also connected to the chamber 36 which is defined by the walls 12, 18 and 20 and by the top wall 16. This chamber 36 is hereinafter referred to as the medium chamber, i.e. a chamber which is arranged to hold a fluid medium.

The chamber 38 which is located below the medium chamber 36 and which extends below the base of the container 24 is hereinafter referred to as the waste chamber. A vent hole (not shown) is provided through the upper part of the outer wall 12 of the waste chamber.

Within the cup-shaped container 24 is positioned a cup-shaped member 40 which has a circumferential outer wall 42, a bottom wall 44, an upstanding inner wall 46, and a top wall 48 which has a central hole 50 therethrough. The bottom wall 44 of cup-shaped member 40 and the base 26 of the container 24 are held spaced apart by a plurality of sector-shaped ribs (not shown) on one or other of the facing surfaces, for example four equispaced ribs. This provides radial passageways 51 between the ribs of for example of the order of 30 microns in depth. The location of the two cup-shaped members 24, 40 in this way leaves a central cylindrical chamber 52 above the hole 34, hereinafter referred to as the insemination chamber, and an outer annular chamber 54, outwardly of the wall 42, hereinafter referred to as the seminal chamber.

As mentioned above, the medium chamber 36 is in communication with the insemination chamber 52 via the hole 34. The passageway between the medium chamber 36 and the insemination chamber 52 is substantially L-shaped, with the valve associated with outlet port 32 being located approximately at the right-angle in the passageway. The valve 35 has a cap 55. In the horizontal portion of the passageway there is located a plastics material rod 56 (FIG. 2) which has a longitudinally extending groove 58 in its peripheral surface, along which the fluid medium from the chamber 36 can pass to the hole 34 and thus into the insemination chamber 52. The groove 58 in the rod serves to control the velocity of the fluid flow from chamber 36 to chamber 52.

The method of using the module of the present invention will now be described. The medium chamber 36 is filled with warm medium using a syringe. The medium must be prepared in advance and equilibrated well in 5% CO2 and at 37° C. Then by attaching a syringe to valve 35 and by exerting suction a flow of the medium from chamber 36 towards the insemination chamber 52 begins. Following the filling of the insemination chamber 52 by medium the module is placed in an incubator at 37° C. and 5% CO2. At the time of follicle aspiration, oocytes are identified and removed from the follicular fluid and possible blood contamination by using a sterile pipette. The oocytes are inserted through hole 50 into the insemination chamber 52 and the module is returned to the incubator.

Approximately 20 minutes is allowed for liquefaction of semen. If the semen does not liquefy it may need to be passed through a 23 gauge needle or a narrow pasture pipette. The semen is laid carefully around the seminal chamber 54. The module is then again placed inside the incubator. During the incubation period, motile sperm move by capillary flow against the flow of fluid. This flow of fluid has already started, from the insemination chamber 52 towards the seminal chamber 54 through the radial passageways 51 (see FIG. 3). Therefore motile sperm approach the insemination chamber. This process happens only for the motile sperm. In other words the non-motile sperm cannot tolerate the rate of flow and cannot reach the insemination chamber. Overflowing medium passes to the seminal chamber. Based on the existence of the membrane filter 28 there will not be any leakage from the seminal chamber to the waste chamber 38. Only seminal plasma passes through the membrane filter 28. Inseminated oocytes are checked for fertilization approximately 15-20 hours after the addition of sperm. For this purpose oocytes are transferred to another dish and cumulous and corona cells are removed from the oocytes using a denudation pipette. After assessment, the fertilized oocytes may continue to be cultured in the module for one further day.

Normal sperm will move against the fluid flow and pass the barrier. This mechanism serves to select the most qualified sperm and only permits the sperm that are capable of moving faster than the flow of fluid to reach the insemination chamber 52.

In the aforementioned method, based on the existence of a continuous flow, any waste products will be continuously washed out and a fresh flow of the medium will be provided at the same time.

The aim of the sperm preparation is to separate the motile sperm from the seminal plasma, non-motile and sluggish sperm, other cellular components and bacteria. It has been shown that all of these factors can have negative effects on fertility. In addition the method of the invention has several advantages over conventional methods:

the present invention utilizes the sperm alignment against flow phenomenon.

the device mimics the migration of sperm through the female genital tract: the seminal chamber acts as a vagina, the passageway 51 between the insemination chamber and the seminal chamber acts as a cervix and the insemination chamber acts as a fallopian tube (fertilization site).

the module does not cause any damage to the sperm, because the procedure does not require any centrifugation or chemicals.

with this method the preparation of sperm as a separate process is not necessary.

the method is rapid and simple.

the process of sperm separation is under direct observation and can be easily controlled. Other methods tend to be blind and there is little or no control while performing the process and it is not until the end of the process that the quality of sperm can be evaluated.

1. An apparatus for insemination of oocytes comprising a multi-chamber module having a first chamber for holding a fluid medium, a second chamber for receiving semen, and a third chamber into which the fluid medium is arranged to flow from the first chamber and from which that fluid medium is arranged to pass into the second chamber, thereby to permit sperm to move against the fluid flow and pass from the second chamber into the third chamber where insemination of ooctyes is arranged to take place:

2. An apparatus according to claim 1, in which the second and third chambers are separated by barrier means through which fluid is arranged to flow from the third chamber to the second chamber and through which sperm are arranged to pass from the second chamber to the third chamber.

3. An apparatus according to claim 2, wherein the barrier means comprises at least one passageway between the second and third chambers.

4. An apparatus according to claim 2, in which the barrier means comprises a pair of confronting surfaces spaced apart by separating means.

5. An apparatus according to claim 4, in which the separating means comprises a plurality of sector-shaped ribs on one or other of the confronting surfaces with passageways therebetween.

6. An apparatus according to claim 5, in which the passageways are of the order of 30 microns in depth.

7. An apparatus according to claim 1, further comprising control means for controlling the velocity of flow of the fluid medium from the first chamber to the third chamber.

8. An apparatus according to claim 7, wherein the control means comprises a rod having a longitudinally extending groove in its peripheral surface, the rod being located within a fluid medium passageway.

9. An apparatus according to claim 1, wherein the second and third chambers are defined by cup-shaped containers, one within the other and separated by an annular flow-restricting barrier.

10. An apparatus according claim 1, further comprising a waste chamber in fluid communication with the second chamber.

11. An apparatus according to claim 10, further comprising filter means located between the second chamber and the waste chamber.

12. A method of in vitro fertilization comprising the steps of:
providing a multi-chamber module comprising a first chamber for holding fluid medium, a second chamber for receiving sperm, and a third chamber within which insemination of oocytes is arranged to take place;
providing fluid medium in the first chamber, establishing a flow of the fluid medium from the first chamber to the second chamber via the third chamber;
depositing one or more oocytes in the third chamber;
depositing semen in the second chamber;
and allowing only motile sperm from the semen in the second chamber to travel to the third chamber against the flow of fluid medium for insemination of the oocytes therein.

13. A method according to claim 12, wherein the multi-chamber module is as defined in any one of claims 1 to 11.

14. A method according to claim 12, further comprising incubating inseminated oocytes in the third chamber.

15. A method according to any of claims claim 12 to 14, wherein there is a steady-state flow of fluid medium from the third chamber to the first chamber.

16. A method of in vitro fertilization which comprises effecting within a single multi-chamber module separation of motile sperm and the insemination of oocytes by the separated motile sperm.
